(19) United States
(12) Patent Application Publication
(10) Pub. No.: US 2014/0058307 A1
(43) Pub. Date: Feb. 27, 2014

(54) ADJUSTABLE BACK BRACE

(71) Applicant: X-Back Bracing Services, Inc., Birdsboro, PA (US)

(72) Inventor: Troy Q. MARSHALL, Blooming Glen, PA (US)

(73) Assignee: X-Back Bracing Services, Inc., Birdsboro, PA (US)

(21) Appl. No.: 13/973,347

(22) Filed: Aug. 22, 2013

Related U.S. Application Data
(60) Provisional application No. 61/692,458, filed on Aug. 23, 2012.

(51) Int. Cl. A61F 5/02

(52) U.S. Cl. CPC A61F 5/02
USPC 602/19

(57) ABSTRACT

A back brace including a back panel, opposed adjustable belt members for encircling a user's abdomen, and at least one dynamic tension strap for applying an adjustable amount of compressive force. The back brace includes a pair of attachment flaps fixedly joined to opposite sides of the back panel. the attachment flaps and belt members have complementary fasteners for mating in a plurality of longitudinal positions to allow adjustment of the belt's length for a broad range of abdomen sizes. The back panel may support a cinch point, and the dynamic tensioning system may include a strap looped through the cinch point and securable to a belt member to apply adjustably compressive force. The cinch points may be supported on straps adjustably fastenable to the back panel in a plurality of relative longitudinal positions to allow for adjustment of dynamic tensioning straps' length for a broad range of abdomen sizes.

### CROSS-REFERENCE TO RELATED APPLICATION

This application claims the benefit of priority under 35 U.S.C. §119(e) of U.S. Provisional Patent Application No. 61/692,458, filed Aug. 23, 2012, the entire disclosure of which is hereby incorporated herein by reference.

### FIELD OF THE INVENTION

The present invention pertains to back braces for creating lift and support of the spine and abdomen. More particularly the present invention relates to an adjustable back brace capable of accommodating a broad range of waist sizes of a wearer of the device, and a variety of back panels spanning different portions of the wearer's back.

### BACKGROUND OF THE INVENTION

There are many medical conditions, including both post-surgical and non-post-surgical conditions, in which it is necessary for a patient to wear a back brace to assist in creating lift and/or support of the spine. Such conditions include compression fractures, herniated discs, bulging discs, severe degenerative disc diseases that cause kyphosis, chronic and acute strain associated with lumbar instability, post-surgical kyphoplasty, mechanical back pain, scoliosis, spondylosis, spondylolisthesis, and post-surgical stabilization.

Many conventional back braces utilize one or more semi-rigid bars that run vertically and/or horizontally across the wearer's torso to which is attached a lower back bracing pad that contacts the wearer's lower back.

Patients generally find the bar or bars across their torso to be uncomfortable. They also often find such back braces uncomfortable due to the lack of airflow to the portions of the body covered by the brace. Even further, conventional back braces apply pressure across the entire area of the back that they cover. However, often patients are bruised in the spinal area or may receive injections adjacent the spine which may make the area surrounding the injection site sore. Conventional back braces that apply pressure directly on the spine, therefore, may further irritate an already sore spinal area or may actually cause pain where there otherwise might be no pain by virtue of the pressure applied directly to the spine.

Conventional lumbar sacral orthotic (LSO), thoracic lumbar sacral orthotic (TLSO) and other orthotic back braces also generally include a semi-rigid back-supporting panel, and are rather complicated to fit and often require several visits to a physician in order to be correctly fitted to the patient. Further, such conventional back braces tend to be static in the sense that, once fitted and worn, the brace is essentially fixed in size for a specific user. More specifically, the semi-rigid supportive panels that are typical of such braces typically need to be trimmed for a smaller-sized back and exchanged for a larger panel for a larger-sized back. Further, to the extent that such braces may have limited adjustability, they typically have such limited adjustability in size that a single brace cannot accommodate multiple different patients having a broad range of different torso sizes, and/or cannot accommodate a single patient as the patient's torso size changes, e.g., while losing or gaining weight. For example, after trimming a semi-rigid panel to a proper size for a patient, the panel will be too small for the patient if the patient gains a significant amount of weight, and such belts often do not allow for accommodating of such increases in weight.

What is needed is an orthotic back brace including a semi-rigid panel that is highly adjustable to adapt to a broad range of abdomen sizes.

### SUMMARY OF THE INVENTION

The present invention provides a back brace including a rigid or semi-rigid (herein, collectively “semi-rigid”) back panel, a pair of opposed belt members for encircling a wearer's torso, and a dynamic tensioning system comprising two dynamic tension straps attached to the back panel that the wearer or a physician may pull forward around the wearer's torso when putting the brace on to tension the straps to achieve a desired level of compressive force on the wearer's back and then attach the free ends of the two straps to the belt members of the brace to maintain that compressive force.

Consistent with the present invention, each back brace includes a pair of attachment flaps fixedly joined to opposite lateral sides of the back panel. Each of the pair of attachment flaps includes a fastener. The brace further includes a pair of opposing belt members for spanning a wearer's abdomen. Each belt member has a complementary fastener for mating with the fastener of one of the pair of attachment flaps in a plurality of longitudinal positions. Accordingly each attachment flap and corresponding belt member together provides a belt portion that is adjustable to allow for shortening or elongating of the length of the belt for accommodating a broad range of abdomen sizes. The belt members further comprise complementary fasteners for mating with each other, to secure the belt around the abdomen.

The back panel may support a point, and the dynamic tensioning system may include a strap secured at one end to the belt member, extending through the cinch point, and then fastenable to the belt member at its opposite end to apply adjustably compressive force. Further, one or more cinch points may be supported on support straps adjustably fastenable to the back panel in a plurality of relative longitudinal positions to allow for shortening and elongating of the effective length of the dynamic tensioning straps for accommodating a broad range of abdomen sizes.

### BRIEF DESCRIPTION OF THE DRAWINGS

An understanding of the following description will be facilitated by reference to the attached drawings, in which:

FIG. 1 is a plan view showing a back of a back brace in accordance with an exemplary embodiment of the present invention;

FIG. 2 is a plan view of the back brace of FIG. 1, showing the adjustable belt members detached from the attachment flaps of the back panel;

FIG. 3 is a partial plan view showing partial detachment of a belt member of the back brace of FIG. 1;

FIG. 4 is a partial plan view showing the belt member repositioned relative to an attachment flap of FIG. 3 to allow for adjustment of the belt member;

FIG. 5 is a partial plan view showing reattachment of the repositioned belt member of FIG. 4;

FIG. 6 is a pictorial of a patient wearing the back brace of FIG. 1, with the opposed belt members encircling the patient's torso;

FIG. 7 is a pictorial of a patient wearing the back grace of FIG. 1, with the dynamic tensioning straps secured to apply compressive force;

FIG. 8-11 are frontal, left rear, rear and right rear views showing how the back brace may be worn in accordance with the principles of the present invention;

FIG. 12 is a plan view showing an inner side of the back brace of FIG. 1, with internal padding removed for illustrative clarity; and

FIG. 13 is a plan view showing interchangeable back panels for use with a single set of opposing belt members.

### DETAILED DESCRIPTION OF THE INVENTION

A back brace in accordance with an exemplary embodiment of the present invention is a lumbar sacral orthotic (LSO) brace 10 providing trunk support extending from the sacral spine to the T-9 vertebrae.

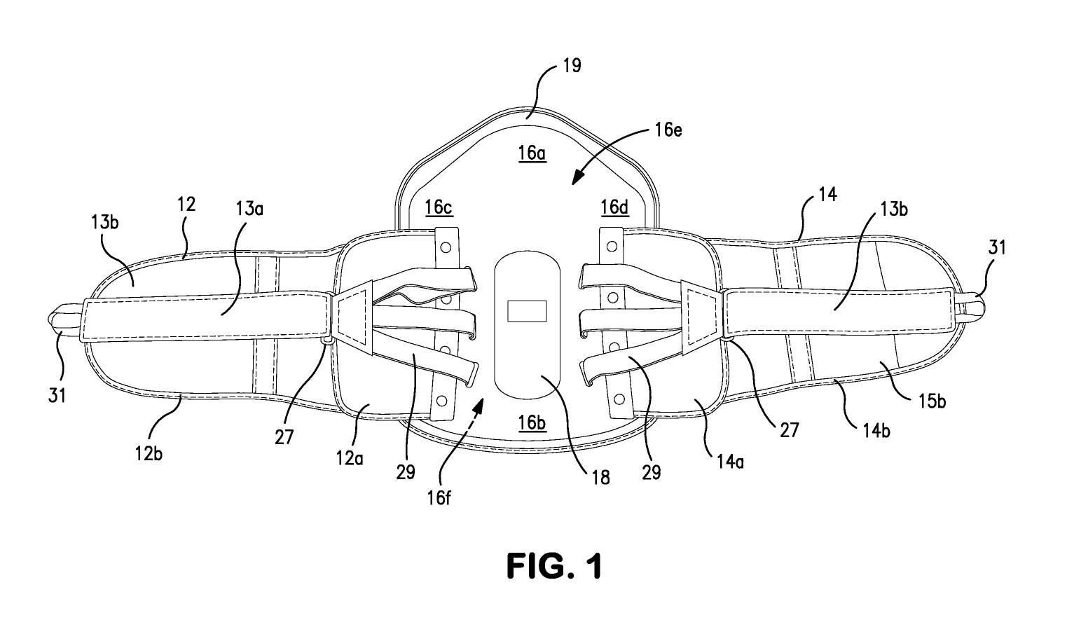

Referring now to FIG. 1, the exemplary back brace 10 comprises a back panel 11 for contacting the wearer's back, opposing adjustable belt members 12, 14 for encircling the wearer's abdomen, and an adjustable dynamic tensioning system 25 comprising two additional straps 13a, 13b.

It should be noted that the use of the term “panel” herein is not intended to imply a necessarily planar shape. In fact, in at least the illustrated embodiment, the back panel 11 has a curved shape closely matching the shape of the human back.

The back panel 11 comprises a rigid or semi-rigid (collectively herein, “semi-rigid”) piece 16, which may, for instance, be formed of 3/32″ thick ABS plastic. As used herein, the term semi-rigid means having a shape, but being elastically deformable or flexible in response to a force. The back piece 16 has a top side 16a, a bottom side 16b, a left lateral side 16c, a right lateral side 16d, an outside major surface 16e, and an inside major surface 16f. The directional terms identified above are for illustrative purposes, in relationship to the body of a person that might wear the back brace. For example, top refers to the side towards the wearer's head, inside refers to the side facing the wearer's body, and left refers to the wearer's left. All directional terms as used herein are relative and not limiting.

In this exemplary embodiment, the piece 16 includes a slot (or cutout) 18 in the middle designed to correspond with the position of the spine when the brace 10 is worn properly. The slot 18 in the semi-rigid back piece 16 has the advantage of eliminating or minimizing direct pressure on the wearer's spine. This will greatly enhance comfort for many wearers insofar as many wearers will have pain or soreness at or near the spine (e.g., from the critical injury, from injections, or from a surgical incision). The exemplary piece 16 provides virtually full support of the back area covered by the back panel 11 without applying pressure directly to the spine over most of its vertical length.

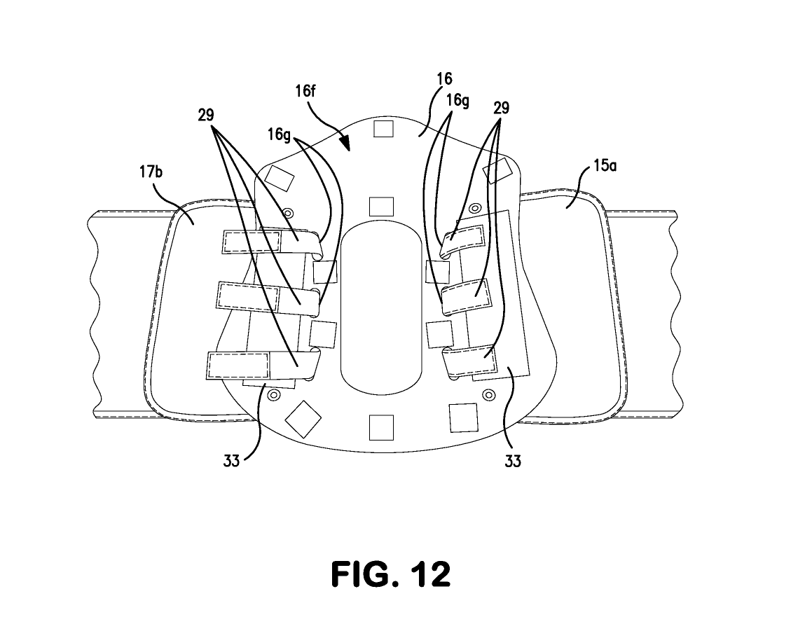

The exemplary back panel 11 further includes soft padding 19, such as Velfoam™ padding, on the inside surface 16f of the piece 16 (i.e., on the side that will contact the wearer). The padding 19 can be formed of or covered with a moisture wicking material to increase wearer comfort. The padding 19 may be attached to the piece 16 by any reasonable means, including a hook and loop attaching system, such as Velcro™, rivets, tape, adhesive, sewing, buttons, snaps, etc., e.g., as shown in FIG. 12. In another embodiment, the padding 19 may be designed with a pocket within which the piece 16 fits.

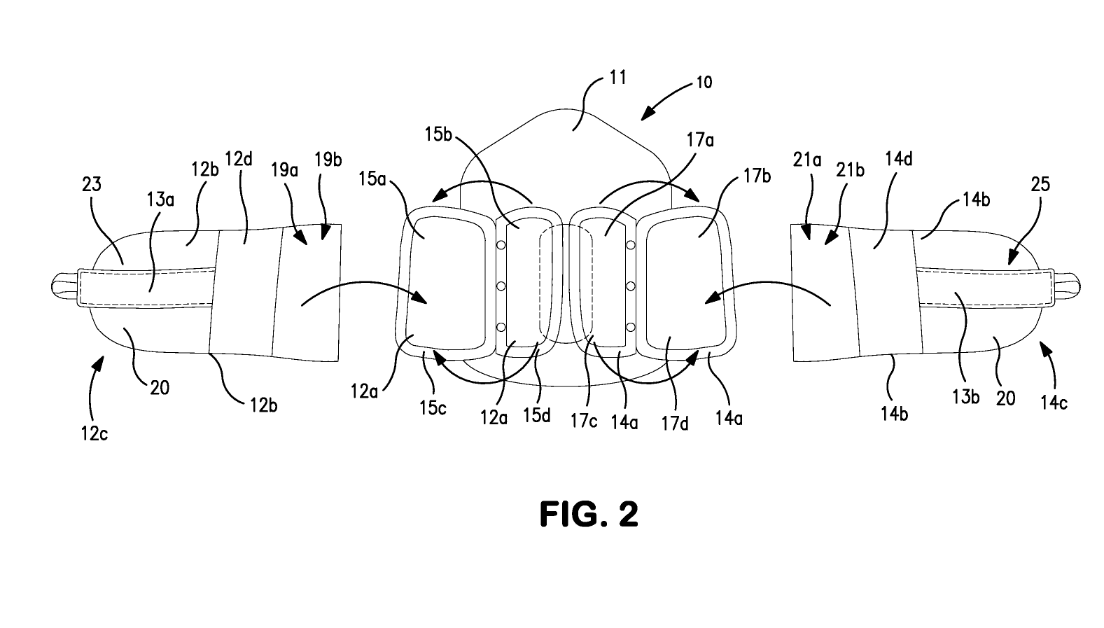

The back panel 11 further includes a pair of attachment flaps 12a, 14a joined to opposite lateral sides 16c, 16d of the back panel 11. The attachment flaps 12a, 14a may be fixedly secured to the back panel 11, e.g., by rivets, as shown in FIGS. 1 and 2. Each of the attachment flaps 12a, 14a comprises a fastener. By way of example, each fastener is preferably a field of hook or loop fastener. Preferably each field is elongated in the longitudinal direction to allow for lateral adjustment of the length of the belt, in that it is longer in the longitudinal direction than is required to make secure and adequate fastening at a plurality of longitudinal positions of a corresponding belt member. By providing elongated fields of fasteners, the belt members 12, 14 may be adjustably secured in a plurality of different relative longitudinal positions. By way of example, providing fields approximately 6 inches in length provides a range of approximately 4 inches of adjustment in the longitudinal direction, to provide about 8 inches of additional range in the abdomen sizes accommodated by the back brace.

The belt members 12b, 14b include fasteners for adjustably attaching to the attachment flaps 12a, 14a. The opposed adjustable belt members 12b, 14b are adjustably attachable to the attachment flaps 12a, 14a. In other words, belt members 12b, 14b may be attached to the attachment flaps 12a, 14a in a plurality of different longitudinal positions. The belt members 12b, 14b are adjustably attachable to the attachment flaps to form a belt portion of variable length, and may be fully detachable from the attachment flaps 12a, 14a, as best shown in FIG. 2.

The fasteners are selected to permit the belt members to be releasably secured and reattached to the attachment flaps 12a, 14a in a plurality of distinctly different relative longitudinal positions, as discussed in greater detail below. In a preferred embodiment, the fasteners include at least one field of one of a hook and loop fastener system, and the attachment flaps include at least one field of the other of the hook and loop fastener system. Any suitable fasteners may be used.

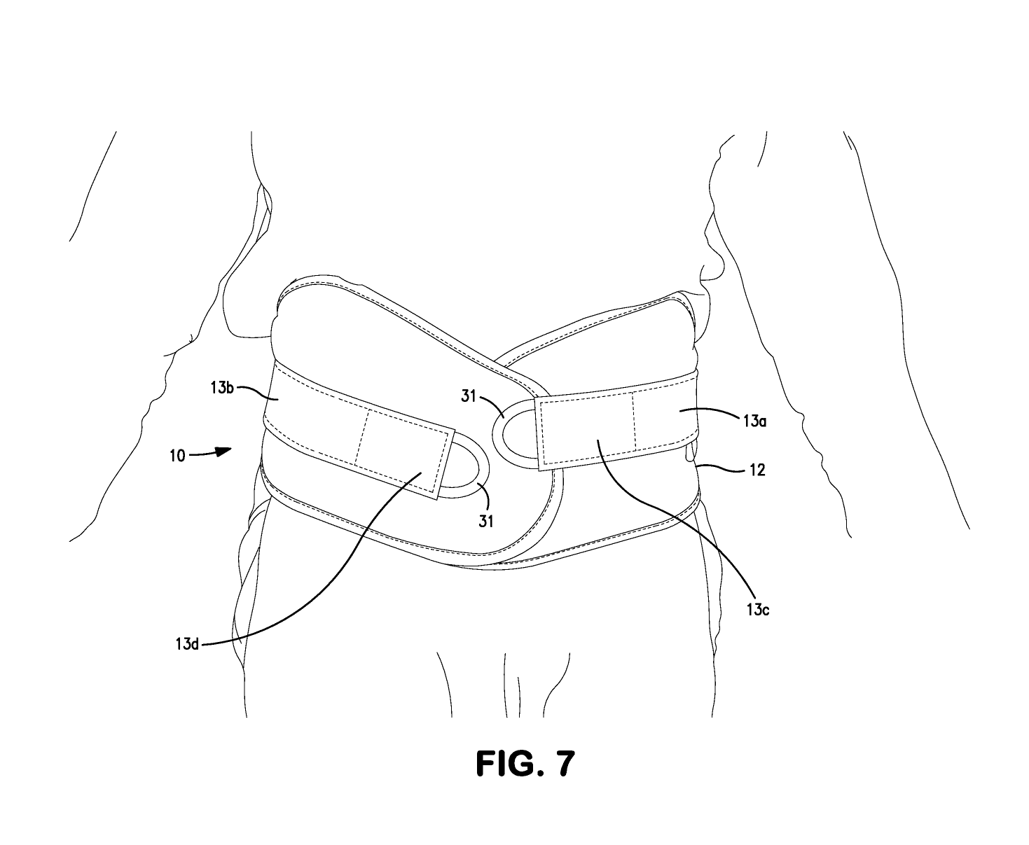

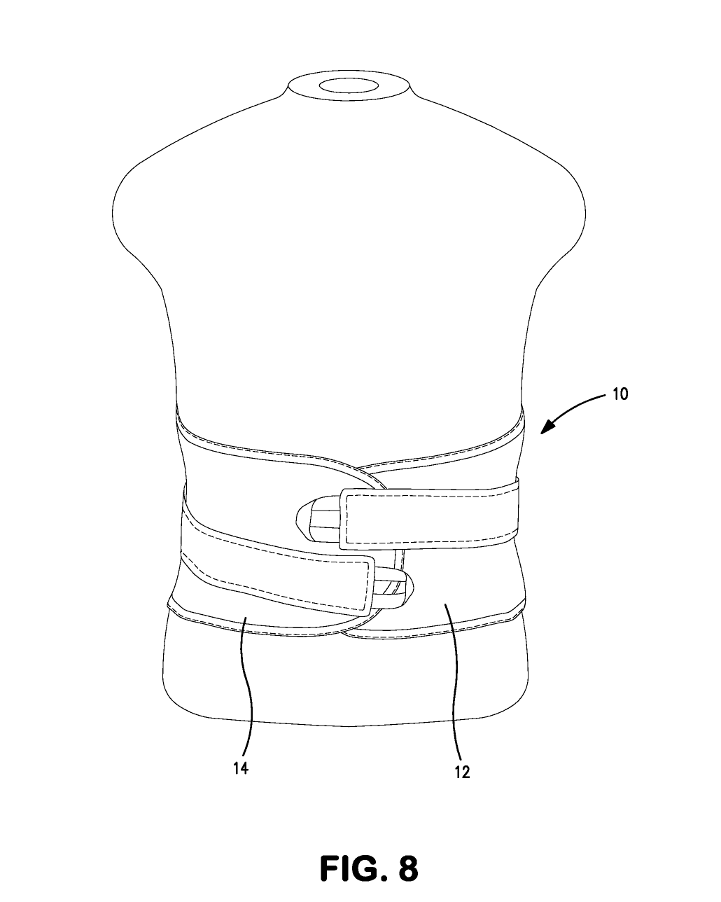

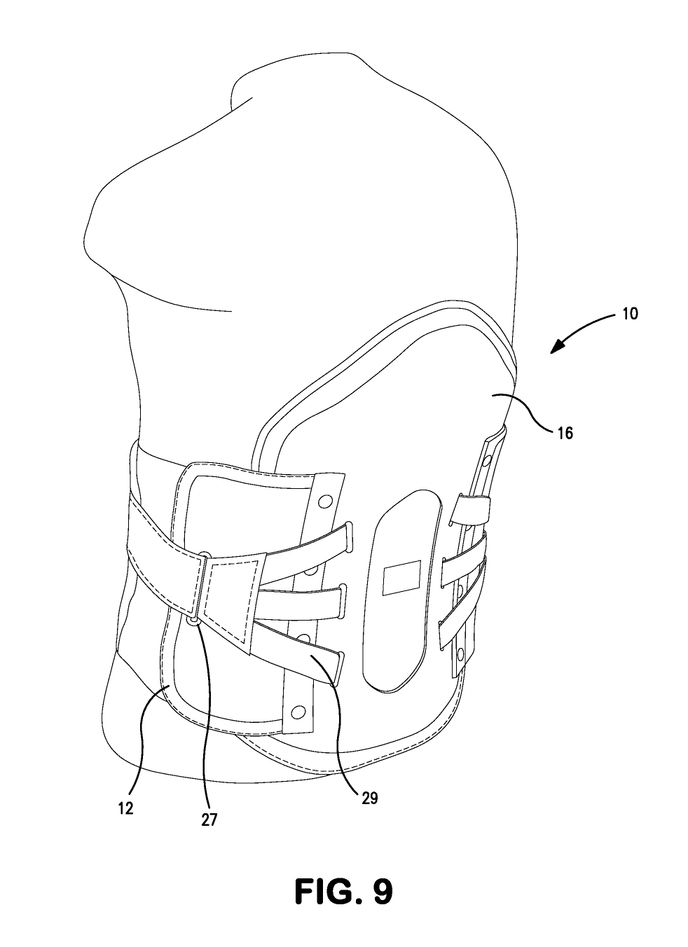

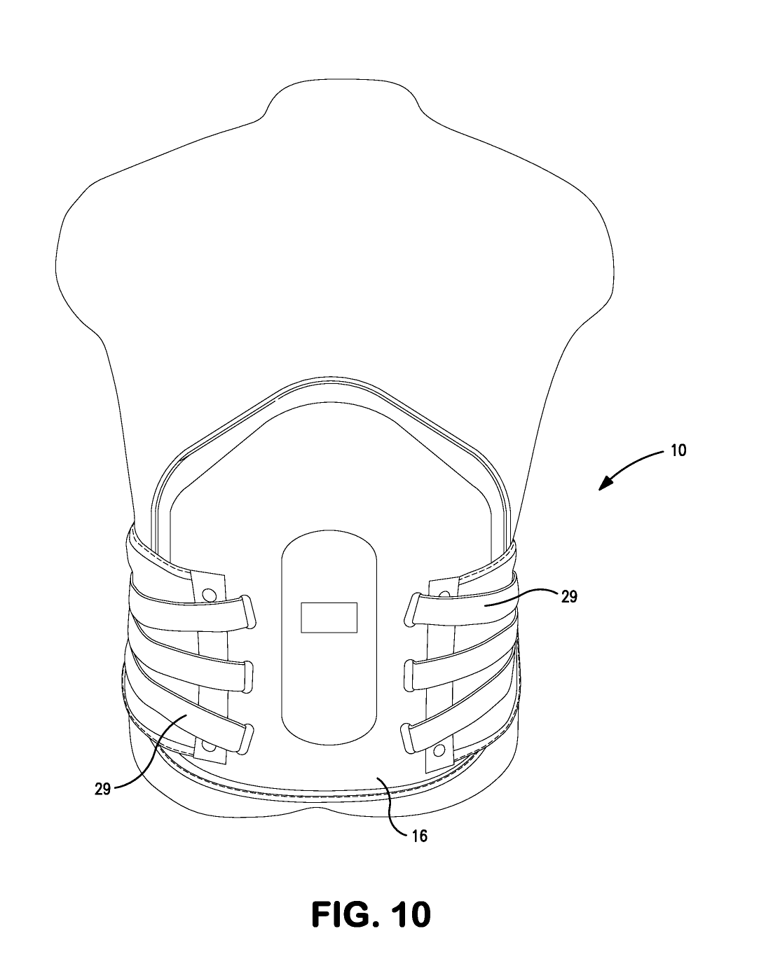

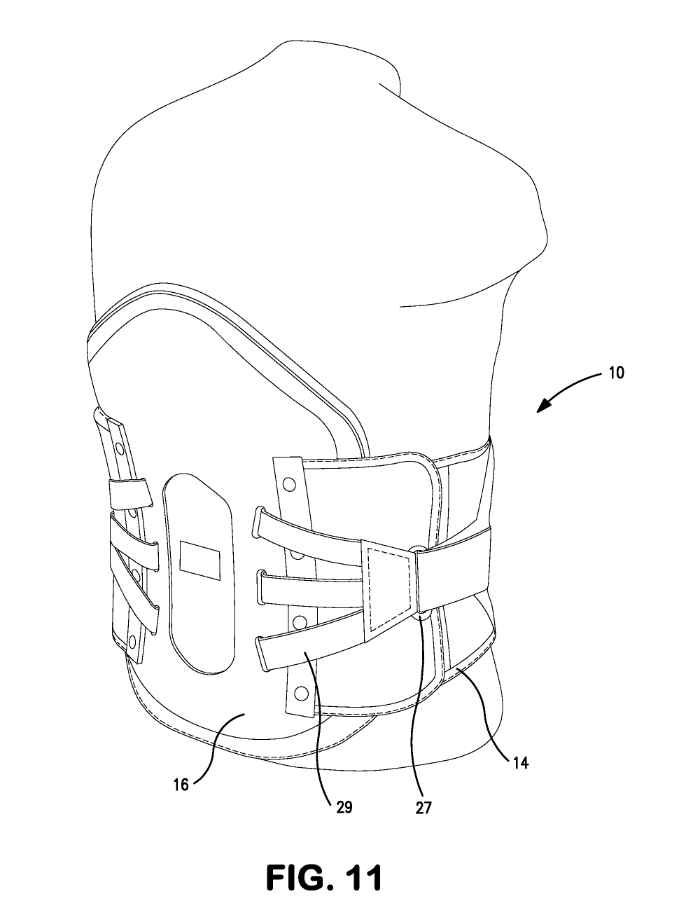

In the exemplary embodiment shown in FIGS. 1-12, each attachment flap, e.g., 12a and 14a, includes a pair of opposed panels, e.g., 15a, 15b and 17a, 17b, each of which is joined to the back piece 16, preferably to its outside major surface 16e. Further, each of the opposed panels includes fasteners, such a fields 15c, 15d, 17c, 17d of hooks, as best shown in FIG. 2. Further, each belt member 12b, 14b includes complementary fasteners, such as a pair of opposed (one on each side of the belt member) fields 19a, 19b, 21a, 21b of complementary loops. Accordingly, a belt member, e.g. 12b, of a belt portion 12 may be attached to an attachment flap, e.g. 12a, of the belt portion 12 by releasably mating the hook and loop fields 15c, 19b and the hook and loop fields 19a, 15d, with the fields 19a, 19b of the belt member 12b sandwiched between the fields 15c, 15d of the corresponding flaps 15a, 15b, as will be appreciated from FIGS. 1-5.

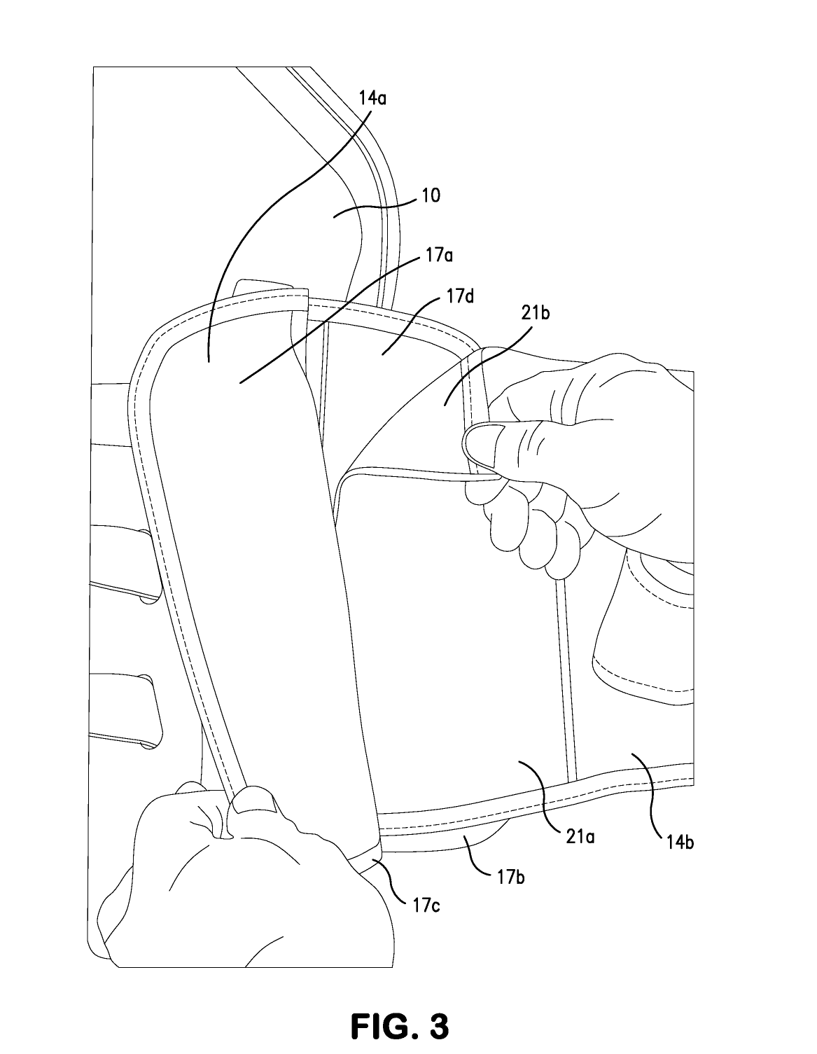

Further, it should be noted that the belt members 12b, 14b are attachable to the attachment flaps 12a, 14a in a plurality of distinctly different relative longitudinal positions. To detach a belt member, e.g. 14b, from an attachment flap, e.g. 14a, one opposed panel 17a of the attachment flap may be peeled back to separate the mated fields of hook and loop fasteners 17c, 21a, as shown in FIG. 3. Next, the belt member 14b may be peeled from the opposing panel 17b to separate the mated fields of hook and loop fasteners 17d, 21b, as best shown in FIG. 3.

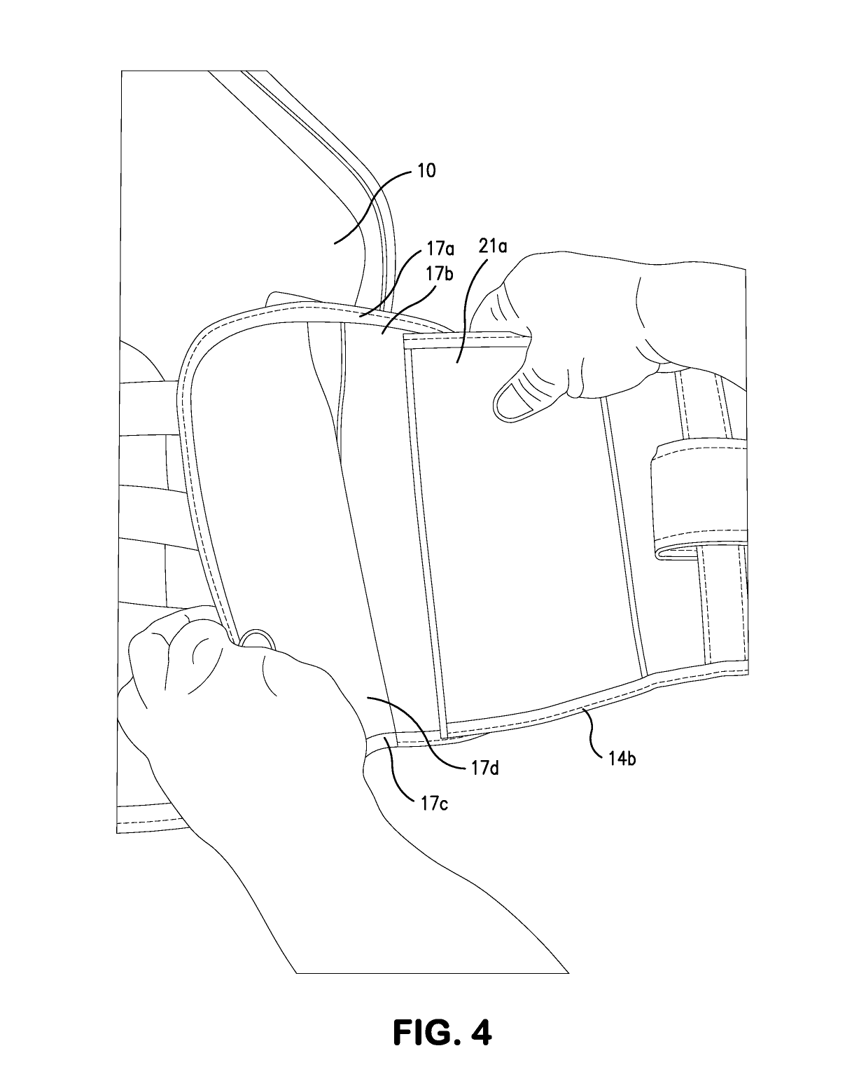

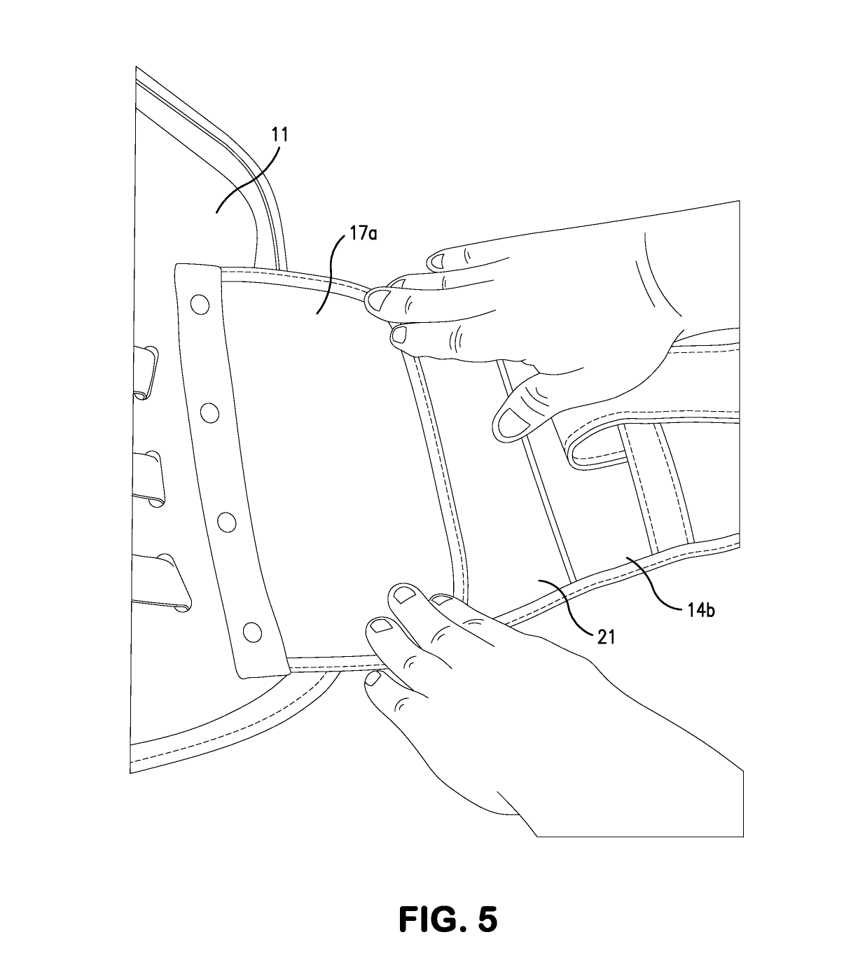

After separation of the belt member 14b from the attachment flap 14a, the belt member may be longitudinally repositioned relative to the attachment flap 14a. For example, the belt member 14b could be moved toward the right in FIG. 4 to lengthen the belt, or toward the left in FIG. 4 to shorten the belt. After repositioning the belt member as desired, the belt member 14b may be secured to a first opposed panel 17b to mate the fields of hook and loop fasteners 17d, 21b, as shown in FIG. 4. Then the second opposed panel 17a may be folded over the belt member 14b to mate the remaining fields 17c, 21a of the second opposed panel and belt member 14b, as shown in FIG. 5.

A similar detachment, adjustment and re-attachment process may be similarly conducted for the opposite belt member 12 on the opposite side 16c of the back piece 16. By way of example, fields approximately 5 inches wide provide approximately 8 inches of adjustment in the longitudinal direction, to provide about 8 inches of range in the abdomen sizes accommodated by the back brace.

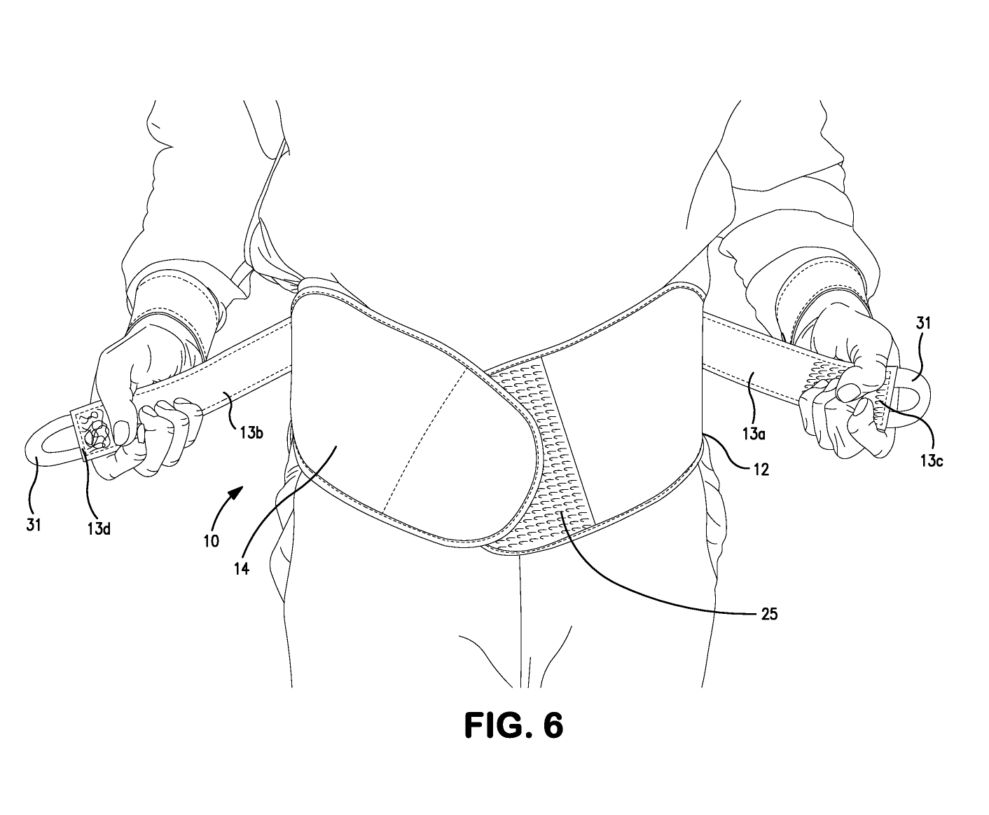

Each belt member 12, 14 has a distal end portion 12c, 14c including complementary fasteners. In the exemplary embodiment shown, hook and loop fasteners are used on opposite sides of the belt members, but any suitable fastener may be used. In the exemplary embodiment shown, distal end portion 12c includes a field of loops 23 on a back side of the distal belt member 12b, and distal end portion 14c of the opposing distal belt portion 14b includes a field of complementary hooks 25 on its front side, as best shown in FIGS. 2 and 6. Accordingly, the back brace may be positioned around a patient's torso, and may be secured across the patient's abdomen by fixing the belt members 12, 14 to each other with the complementary fields of fasteners 23, 25 mated, as best shown in FIG. 6. By providing relative wide fields of fasteners, the belt members 12, 14 may be adjustably secured in a plurality of different relative longitudinal positions. By way of example, providing fields 23, 25 approximately 4 inches in width provides a range of approximately 6 inches of adjustment in the longitudinal direction, to provide about 6 inches of additional range in the abdomen sizes accommodated by the back brace.

Further, each of belt members 12, 14 may include an elastic panel 12d, 14d that is readily stretchable in the longitudinal direction, as shown in FIG. 2. This elastic panel 12d, 14d is not only useful in accommodating movements of the patient and maintaining the brace in place after affixing the back brace, but is also useful in providing for additional adjustment of the belts. By way of example, elastic panels providing approximately 2 inches of elongation in the longitudinal direction provides a range of approximately 4 inches of adjustment in the longitudinal direction, to provide about 4 inches of additional range in the abdomen sizes accommodated by the back brace. Accordingly, the exemplary embodiment is suitable for accommodating a broad range of abdomen sizes, e.g., in the range of about 32-50 inches.

Each of the belt members 12, 14 may further include panels of soft padding 20, such as ¼″ Velfoam™, toward their distal end portions 12c, 14c of a size and shape suitable for wrapping around the wearer's abdomen, as best shown in FIG. 2. In one embodiment, a belt member 12 includes a pocket for accepting a semi-rigid plastic piece that can be inserted in the pocket to provide semi-rigidity to the belt portion, if desired. In one embodiment, the plastic piece also is 3/32″ ABS plastic. Two flaps bearing one of the hook or loop surfaces of a hook and loop attaching system may be provided for at least partially closing the opening of the pocket so that the plastic piece cannot fall out inadvertently, but can be readily removed upon releasing of the flaps.

The two dynamic tensioning straps 13a, 13b extend from the belt members, as best shown in FIGS. 1 and 2. In this embodiment, dynamic tensioning straps 13a, 13b are permanently fixed to the belt members by stitching, and are inelastic, though in other embodiments the straps may be elastic and/or detachably affixed. Each of the straps 13a, 13b passes through a cinch point, such as a ring 27, as best shown in FIGS. 1, 9 and 11. Each cinch point is supported on the back panel.

In the example shown, each cinch point is supported by support strapping 29 passing through openings 16g in the back piece 16 and being joined to its inner surface 16f. The exemplary support strapping 29 is adjustably positionable longitudinally to provide for longitudinal adjustment of the cinch point 27. In the exemplary embodiment such adjustability is provided for by adjustably and repositionably securing the strapping to the back piece 16 by way of hook and look attachment structures, as best shown in FIG. 12. Preferably, the hook and loop structures are provided in relatively wide fields 33 to provide for adjustable positioning of the support strapping 29 relative to the piece 16. For example, a 2-inch wide field of fastener on the back piece 16 provides for about 2-inches of longitudinal adjustment for each dynamic tensioning strap, for a total of 4 inches of adjustment for tensioning purposes, as best shown in FIG. 12. In the exemplary embodiment shown in FIG. 12, the support strapping 29 on the left side of the back piece 16 is fastened in a position for longitudinally shortening the reach of the corresponding dynamic tensioning strap, and the support strapping 29 on the right side of the back piece 16 is fastened in a position for longitudinally lengthening the reach of the corresponding dynamic tensioning strap, for illustrative purposes. Further, the support strapping 29 may be constructed of elastic materials to allow for movements of the wearer and/or accommodate different abdomen sizes.

Once the wearer is wearing the back brace as just described, the dynamic tensioning system 25 can now be used to easily adjust the brace 10 to provide the desired level of compressive force on the wearer's torso in order to provide the desired compression on the wearer's back. In the illustrated embodiment, the dynamic tensioning system 25 comprises two essentially identical support strap assemblies 13a, 13b. In the particular embodiment illustrated in the drawings, each strap assembly comprises a non-elastic webbing looped through a cinch point 27 supported by elastic (preferably, semi-elastic) strapping 29. As used herein, the term semi-elastic refers to the property of stretching in response to increasing force over a certain range and then becoming inelastic upon reaching a predetermined force. In other embodiments, the elasticity or inelasticity of these various components may be changed or interchanged.

In any event, returning to the particular illustrated embodiment, the dynamic tensioning straps 13a, 13b include one of the hook and loop fields 34 of a hook and loop attaching system at least near their distal ends 13c, 13d for purposes of enabling removable attachment of the distal ends 13c, 13d of the straps 13a, 13b to the belt members 12, 14 of the back brace 10, best shown in FIGS. 1, 6 and 7, and as described in more detail further below. Further each strap 13a, 13b may include a loop 31 near the distal ends 13c, 13d, which can be used by the patient during tensioning to grasp and pull the straps 13a, 13b, e.g., by looping the thumb of each hand through a respective loop 31.

As can be seen in the drawings, the dynamic tension straps 13a, 13b are permanently attached to the belt members 12, 14. This is a desirable feature in some cases because it reduces the possibility of losing the dynamic tension straps 13a, 13b. Attachment may be achieved in any reasonable fashion. In the illustrated embodiment, the straps are and sewn to the belt members 12, 14. In other embodiments, the dynamic tension straps 13a, 13b may be directly attached to the belt members by any other suitable means, such as rivets, screws, adhesive, snaps, buttons, buckles, hook and loop systems, etc.

When the wearer is wearing the brace 10 as previously described, whereby it is wrapped around the wearer's torso and the two belt members 12, 14 are attached to one another to form a complete loop around the wearer's torso, the wearer (or a third person, such as a physician or physician's assistant) can grab one of the dynamic tension straps 13a, 13b in each hand, as shown in FIG. 6, and pull them forward to pull the back panel 11 tighter to the wearer's back (i.e., compress the wearer's back). When the desired tension is reached, the distal ends 13c, 13d of the dynamic tension straps 13a, 13b may be attached to the belt members 12, 14 of the brace 10 to maintain the compression force after the wearer (or third person) lets go of the dynamic tension straps 13a, 13b. In this particular embodiment using a hook and loop attachment system, the wearer merely needs to press the distal ends 13c, 13d of the dynamic tension straps 13a, 13b against the belt members 12, 14 and they will be attached thereto by means of the hook and loop attaching system. The hook and loop attaching system allows easy attachment and detachment for readjusting the compression force and/or taking the back brace 10 on and off.

The use of mating hook and loop surfaces on the straps 13a, 13b and outside surface belt members 12, 14 is particularly useful because it is both easy to attach and detach and is essentially infinitely adjustable. However, there are any number of other ways to provide adjustable attachment of the dynamic tension straps 13a, 13b to the belt members. For instance, each strap 13a, 13b may be provided with both the hook and the loop surface on different parts of the strap assembly and the belt members may have a square ring or other loop that the distal ends 13b, 15b of the strap assembly can be passed through and then the strap assembly may be folded over on itself to attach the hook and loop portions to each other. Additional possibilities include providing buckles on the belt members for adjustably locking the straps at any desired tension.

In one embodiment of the invention, the elastic strapping 29 is semi-elastic and reaches maximum stretch at 200 pounds of force.

When the dynamic tension system 25 is deployed as described above, it may relieve some or all of the tension in the belt members 12, 14. Hence, the dynamic tension straps 13a, 13b may primarily or exclusively provide the compression force on the wearer's back. The compression provided by the elasticity of the dynamic tension straps 13a, 13b provides dynamic tension on the wearer's back, providing several advantages. For instance, it allows the back brace 10 to adapt in accordance with twisting and other motions of the wearer without restricting the wearer's movement and without causing the back panel 11 to lose contact with the wearer's back.

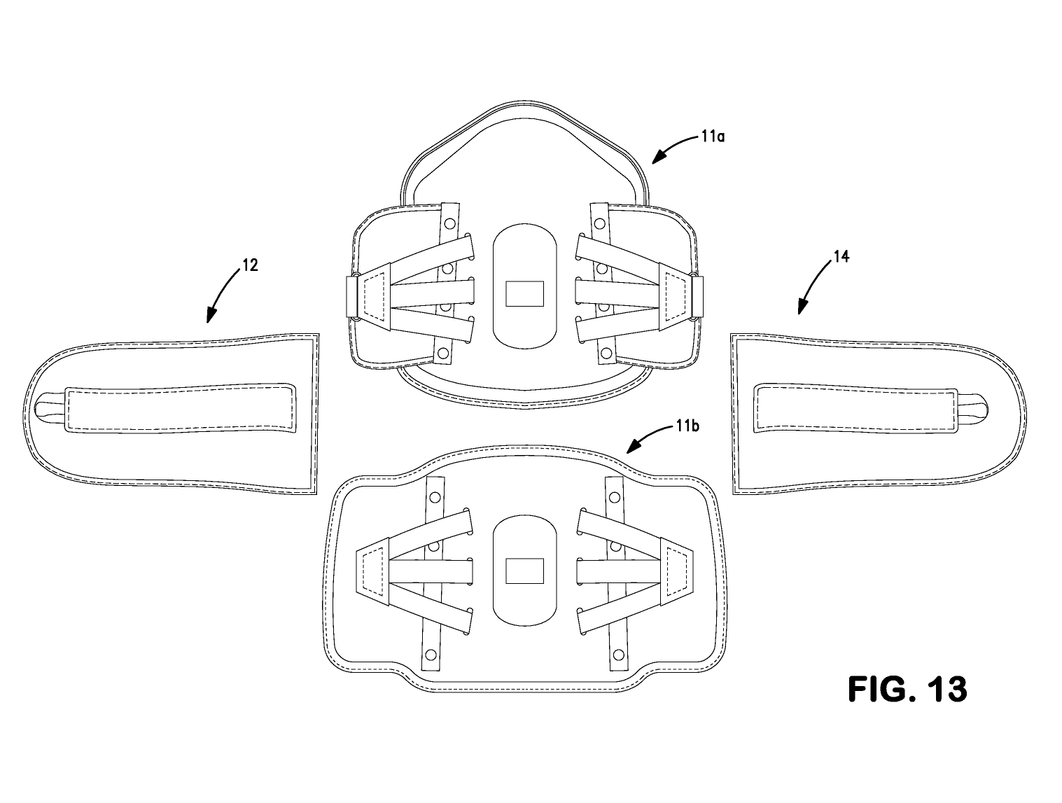

As noted above, the exemplary back brace is a lumbar sacral orthotic (LSO) brace providing trunk support extending from the sacral spine to the T-9 vertebrae. However, the invention can be incorporated into back braces of other sizes, such as full-sized LSO back braces that provide trunk support extending from the sacral spine to the scapular plane, or a TLSO back brace providing support from the sacral spine to the scapular plane. Generally, the type of brace and corresponding support is largely a function of the extent of the spine spanned by the back brace's back panel, as discussed herein. FIG. 13 shows that not only may be brace 10 be configured with back panels 11a, 11b of various sizes, but the brace is preferably constructed to permit back panels of different sizes to be used interchangeably with a single set of belt member 12, 14.

The back brace of the present invention is simple to fit to wearers of widely varied ages and sizes, and to adjust to a single wearer as the wearer's abdomen size changes, e.g., when losing or gaining weight. However, it also may be provided in different sizes in order to better accommodate different sized wearers. For example, consistent with the teachings herein braces could be manufactured in two adjustable sizes, such as size A with a semi-rigid back panel for a 19 inch waist to a 40 inch waist and a (correspondingly adjustable belt), and size B with a semi-rigid back panel for a 41 inch waist to a 60 inch waist (and a correspondingly adjustable belt). In particular, the size and/or shape of the back panel 11 can be altered to accommodate different sized wearers. Accordingly, the back brace are highly adjustable to adapt to a broad range of abdomen sizes, e.g., providing a range of at least 10 inches of circumferential adjustment, and preferably at least 15 inches of circumferential adjustment, and most preferably at least 20 inches of circumferential adjustment. In preferred embodiments, circumferential adjustment is enabled not only in the belt members, but also in the dynamic tension straps. In addition, the back brace can be offered in different sizes for supporting and/or hyper-extending different portions of the spine, including standard LSO, full LSO, and TLSO sizes.

Furthermore, skin breakdown is reduced due to the slot 18 in the back piece 16. The brace is easy to use and is light in weight, providing consistent compression and pain relief.

The brace 10 can be used post-operatively and the semi-rigid back piece 16 may be made heat moldable in order to achieve a more custom fit, if necessary.

The ambulatory wearer may begin a conservative exercise program, thus allowing the wearer to strengthen the supportive muscular system, thereby promoting independence and a return to normal everyday activities.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

1. A back brace comprising:
a back support for placement against a wearer's back, the back support comprising:
a semi-rigid back panel; and
a pair of attachment flaps joined to opposite lateral sides of said back panel, each of said pair of attachment flaps comprising a fastener;
a pair of opposing belt members for spanning a wearer's abdomen, each pair of opposing belt members comprising an attachment flap and a belt member, each attachment flap comprising a complementary fastener for mating with said fastener of one of said pair of attachment flaps in a plurality of longitudinal positions relative, said belt members comprising complementary fasteners for mating with each other.

2. The back brace of claim 1, further comprising:
a cinch ring supported on said back panel; and
at least one dynamic tension strap joined at one end to at least one of said belt members, said belt member passing through said cinch ring and being releasably securable at an opposite end to at least one of said belt portions in a plurality of longitudinal positions so as to compress the wearer's torso.

3. The back brace of claim 2, wherein each dynamic tension strap comprises non-elastic webbing.

4. The back brace of claim 2, further comprising a pair of cinch rings, each of said pair of cinch rings being supported on a support strap fastened to said back panel.

5. The back brace of claim 4, wherein said back panel defines a pair of openings, and wherein each of said support straps passes through a respective one of said pair of openings.

6. The back brace of claim 4, wherein each support strap comprises semi-elastic strap material.

7. The back brace of claim 2, further comprising a pair of cinch rings, each of said pair of cinch rings being on a strap assembly comprising a plurality of support straps, each support strap being fastened to said back panel.

8. The back brace of claim 7, wherein said back panel defines a plurality of openings, and wherein each of said support straps passes through a respective one of said plurality of openings.

9. The back brace of claim 7, wherein said back panel comprises one fastener of a hook and look fastener system, and each support strap comprises the other fastener of a hook and loop fastener system, and wherein each of said support straps is removably fastenable to said back panel in a plurality of relative longitudinal positions to allow for shortening and elongating of the effective length of the dynamic tensioning straps.

10. The back brace of claim 1, wherein said back panel comprises a semi-rigid piece defining a central slot positioned to receiving the spine of a wearer of the back brace.

11. The back brace of claim 1, wherein each of said pair of attachment flaps is fixedly joined to said back panel.

12. The back brace of claim 1, wherein each of said pair of attachment flaps comprises a pair of opposed panels.

13. The back brace of claim 1, wherein each of said pair of opposing belt members comprises an elastic panel stretchable in the longitudinal direction.

14. The back brace of claim 1, wherein at least one of said pair of opposing belt members comprises a pocket for accepting a semi-rigid plastic piece that can be inserted in the pocket to provide semi-rigidity to the belt portion.

15. A back brace kit comprising:
a first back support of a first size for placement against a wearer's back; and
a second back support of a second size different from said first size for placement against the wearer's back;
each back support comprising:
a semi-rigid back panel; and
a pair of attachment flaps joined to opposite lateral sides of said back panel, each of said pair of attachment flaps comprising a fastener; and
a pair of opposing belt members for spanning a wearer's abdomen, each pair of opposing belt members comprising an attachment flap and a belt member, each attachment flap comprising a complementary fastener for mating with said fastener of one of said pair of attachment flaps in a plurality of longitudinal positions relative, said belt members comprising complementary fasteners for mating with each other.

16. The back brace kit of claim 15, wherein each of said first and second back supports comprises:
a cinch ring supported on said back panel; and
at least one dynamic tension strap joined at one end to at least one of said belt members, said belt member passing through said cinch ring and being releasably securable at an opposite end to at least one of said belt portions in a plurality of longitudinal positions so as to compress the wearer's torso.

17. The back brace kit of claim 15, wherein each of said first and second back supports comprises:
a pair of cinch rings, each of said pair of cinch rings being supported on a support strap fastened to said back panel; and
at least one dynamic tension strap joined at one end to at least one of said belt members, said belt member passing through said cinch ring and being releasably securable at an opposite end to at least one of said belt portions in a plurality of longitudinal positions so as to compress the wearer's torso.

18. The back brace kit of claim 17, wherein each of said pair of cinch rings is supported on a strap assembly comprising a plurality of support straps, each support strap being fastened to said back panel.

19. The back brace kit of claim 18, wherein each back panel defines a plurality of openings, and wherein each of said plurality of support straps passes through a respective one of said plurality of openings.

20. The back brace kit of claim 19, wherein each back panel comprises one fastener of a hook and look fastener system, and each support strap comprises the other fastener of a hook and loop fastener system, and wherein each of said support straps is removably fastenable to a respective back panel in a plurality of relative longitudinal positions to allow for shortening and elongating of the effective length of the dynamic tensioning straps.
